(19) United States
(12) Patent Application Publication
(10) Pub. No.: US 2025/0083116 A1
(43) Pub. Date: Mar. 13, 2025

(54) SYSTEMS AND METHOD FOR RETROFITTING BROWNFIELD PLANTS

(71) Applicant: NiQuan Energy, LLC, Washington, DC (US)

(72) Inventor: Ainsley Gill, Washington, DC (US)

(73) Assignee: NiQuan Energy, LLC, Washington, DC (US)

(21) Appl. No.: 18/593,764

(22) Filed: Mar. 1, 2024

Related U.S. Application Data
(60) Provisional application No. 63/538,061, filed on Sep. 12, 2023.

(51) Int. Cl. B01J 19/00; B01D 3/14; B01D 53/14; B01D 53/22; B01J 19/24; C01B 3/38; C01B 3/50; C12M 1/00; C12M 1/04; C12M 1/34

(52) U.S. Cl. CPC B01J 19/002; B01D 3/145; B01D 53/1475; B01D 53/229; B01J 19/0013; B01J 19/006; B01J 19/245; C01B 3/382; C01B 3/501; C12M 27/04; C12M 41/30; C12M 43/04; B01D 2252/20478; B01D 2256/16; B01D 2257/304; B01D 2257/504; B01J 2219/00024; B01J 2219/00087; B01J 2219/00164; B01J 2219/00186; B01J 2219/00265; C01B 2203/0233; C01B 2203/0405; C01B 2203/0475; C01B 2203/061; C01B 2203/062; C01B 2203/068; C01B 2203/0883; C01B 2203/1241; C01B 2203/127; C01B 2203/142; C01B 2203/1638

(57) ABSTRACT

The invention modernizes abandoned or inefficient petrochemical plants for the production of jet fuel, diesel, naphtha, drilling fuels and wax. It utilizes an amine system shifted in a brownfield situation and a PRISM unit to cleanse incoming syngas and obtain an optimal H2:CO ratio for conversion. The refit and repurposing introduces novel heat transfer elements to a Fischer-Tropsch (FT) reactor and embarks a proprietary FT catalyst for the production of GTL products. It also incorporates unique FT analyzers to monitor hydrocarbon streams and aid production and Coriolis flow meters for precise measurements of liquid wax flow, unaffected by wax congestion or vibration. Feedstocks include numerous sources such as Natural gas, Biomass, etc

### CROSS-REFERENCE TO RELATED APPLICATIONS

None.

### BACKGROUND

### Field of Art

The present invention relates to energy production and refitting brownfield plants to improve efficiency and lessen environmental impacts.

### Discussion of the State of the Art

In the realm of biodegradable fuel production, there are generally two types of commercial plants: brownfield and greenfield. Greenfield plants are newly constructed facilities that often come with the advantage of incorporating the latest technologies and efficiencies. However, the construction of greenfield plants requires significant capital expenditure, making them a costly option for fuel production.

On the other hand, brownfield plants are idle or under-utilized industrial sites that have been used previously. These stranded assets include stranded gas fields and/or mothballed petrochemical plants, which represent untapped resources that are often left unused due to various economic or logistical constraints. These assets are particularly prevalent in the methanol and ammonia petrochemical industries. While these assets may be available, their potential for contributing to energy production and environmental sustainability has been largely overlooked because brownfield plants present their own set of challenges, particularly when it comes to updating and retrofitting them for modern energy production needs. As of yet, no one has been able to successfully retrofit brownfield plants to create biodegradable fuels and products, including, but not limited to jet fuel, diesel, naphtha drilling muds, LPG and 2 wax in commercial quantities, while meeting modern environmental standards, because transforming such locations into efficient fuel-producing plants is technically challenging and costly. Attempts to mitigate these issues have abounded but with limited success. Currently available solutions have failed to deliver meaningful improvements, often only producing marginal increases in fuel yield. Despite this, they have invariably escalated costs and raised concerns due to the lengthy payback period leading to questionable economic viability.

Simultaneously, there is an increasing demand for clean, biodegradable products in sectors that are hard to abate, such as aviation, heavy industry, and shipping. These sectors are significant contributors to greenhouse gas (GHG) emissions and are often reliant on fossil fuels. But there are no available solutions currently on the market that are environmentally sustainable but also commercially viable.

Moreover, the energy sector faces the issue of gas flaring, a practice that not only wastes valuable resources but also contributes to GHG emissions. Currently solutions do not reduce the amount of gas flaring, which is crucial for both economic efficiency and environmental protection.

### SUMMARY

The present invention provides a method for repurposing defunct or inefficient production plants that may include petrochemical, methanol and ammonia plants to produce clean biodegradable fuels and products, including, but not limited to jet fuel, diesel, naphtha drilling muds, LPG and wax. This is accomplished through the integration of various modified systems configured together in a novel way, including an amine system, prism units, thermocouples, and refitted Fischer-Tropsch (FT) reactors.

The amine system is adapted for use in a brownfield environment and serves to remove acid gasses from natural gas. This system works in conjunction with other plant systems to purify syngas before it is processed in the PRISM unit. The PRISM unit is configured to achieve a specific hydrogen (H2) to carbon monoxide (CO) ratio, which is essential for optimal conversion of feed syngas into FT wax or syncrude. The invention accommodates a variety of feedstocks, such as natural gas, biomass, renewable natural gas, hydrogen, municipal waste, and flared or stranded gas.

The FT reactors are equipped with specially designed heat transfer elements that are tailored for use in repurposed old ammonia and methanol plants. These reactors employ a unique FT catalyst for the production of Gas-to-Liquids (GTL) products. Additionally, FT analyzers are incorporated to provide real-time analysis of key hydrocarbon streams, facilitating adjustments to maximize production. This modification will help increase efficiencies within the system and maximize the lifespan of the various components, notably the catalyst within the FT reactor.

The invention also includes specialized flow meters that are designed to resist clogging from the wax produced in the process. These meters eliminate vibrations and gaseous medium in piping, allowing for accurate measurement of liquid wax flow. This helps maintain flow throughout the system and reduce the chance of wax hardening and damaging the system.

The invention further includes carbon/gas capture, energy capture, and water recovery systems throughout the plant to help improve the environmental impact of the refitted plant. Carbon/gas capture can be used to release lower amounts of CO2 and CO into the environment as well as capture and produce H2 at a higher purity than can be normally produced at a non-specialized plant. Energy capture can be conducted at the flues and heat recovery areas in the plant to use to either heat water to create steam for other areas of the plant or drive turbines to produce energy used in the rest of the plant. Lastly, water recovery systems are able to recover water that normally would be wasted in a plant that is less efficient.

By incorporating these elements, the invention offers a method for revitalizing abandoned or inefficient fuel production plants. It introduces a cost-effective approach to jet fuel, diesel naphtha, drilling fluids and wax production and is compatible with existing fuel transportation methods.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a GTL wax plant design in an embodiment of the present invention

FIG. 2 illustrates a design for a feed treatment section within a GTL wax plant in an embodiment of the present invention

FIG. 3 illustrates a design for a steam methane reformer section within a GTL wax plant in an embodiment of the present invention

FIG. 4 illustrates a design for a syngas and heat recovery section within a GTL wax plant in an embodiment of the present invention

FIG. 5 illustrates a design for a carbon dioxide removal section within a GTL wax plant in an embodiment of the present invention

FIG. 6 illustrates a design for a hydrogen removal section within a GTL wax plant in an embodiment of the present invention

FIG. 7 illustrates a design for a FT reactor section within a GTL wax plant in an embodiment of the present invention

FIG. 8 illustrates a design for a hydrocracker and stripper section within a GTL wax plant in an embodiment of the present invention

FIG. 9 illustrates a design for a fractionation system section within a GTL wax plant in an embodiment of the present invention

FIG. 10 illustrates a design for a control system within a GTL wax plant in an embodiment of the present invention

FIG. 11 illustrates a design for a liquid-liquid separator within a GTL wax plant in an embodiment of the present invention

FIG. 12 illustrates a design for a product separator within a GTL wax plant in an embodiment of the present invention

FIG. 13 illustrates a method for retrofitting a brownfield plant into a GTL wax plant in an embodiment of the present invention

### DETAILED DESCRIPTION OF EMBODIMENTS

The present invention is for a method for retrofitting brownfield plants. The invention is described by reference to various elements herein. It should be noted, however, that although the various elements of the inventive apparatus are described separately below, the elements need not necessarily be separate. The various embodiments may be interconnected and may be cut out of a singular block or mold. The variety of different ways of forming an inventive apparatus, in accordance with the disclosure herein, may be varied without departing from the scope of the invention.

Generally, one or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices and parts that are connected to each other need not be in continuous connection with each other, unless expressly specified otherwise. In addition, devices and parts that are connected with each other may be connected directly or indirectly through one or more connection means or intermediaries.

A description of an aspect with several components in connection with each other does not imply that all such components are required. To the contrary, a variety of optional components may be described to illustrate a wide variety of possible embodiments and in order to more fully illustrate one or more embodiments. Similarly, although process steps, method steps, or the like may be described in a sequential order, such processes and methods may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, or method is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art.

### System

The apparatus of the present invention is comprised of the following elements illustrated below. The elements individually or in combination provide the benefits described above.

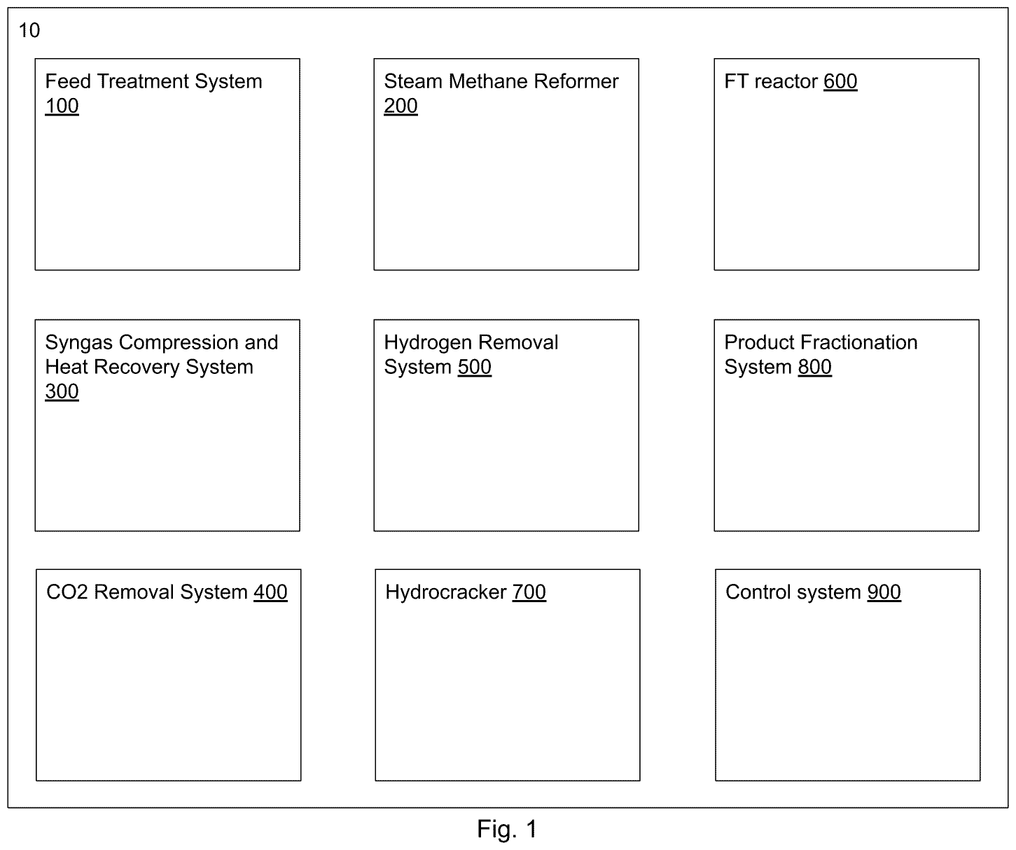

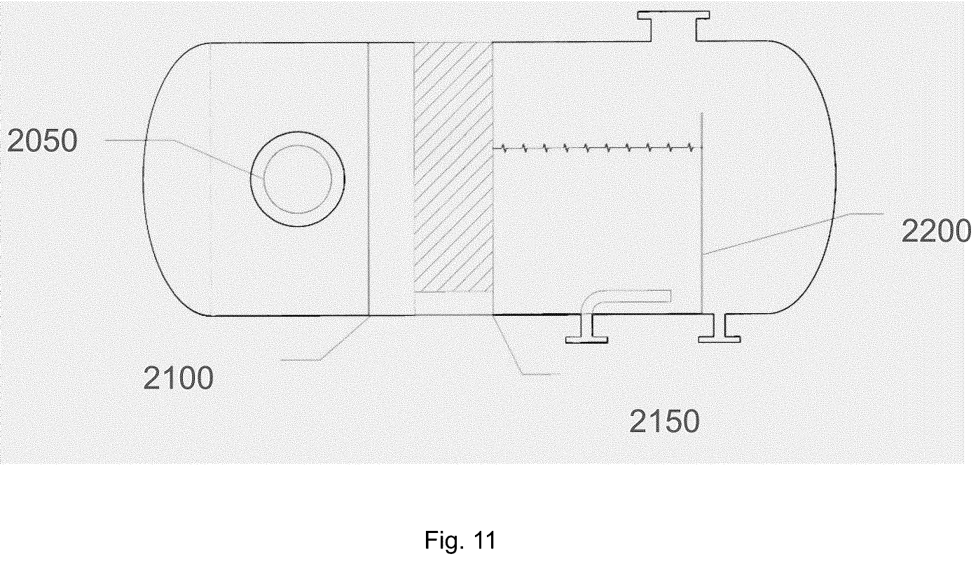

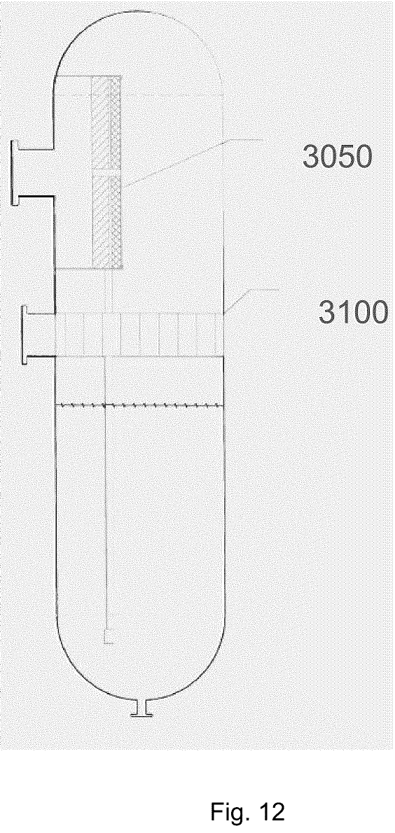

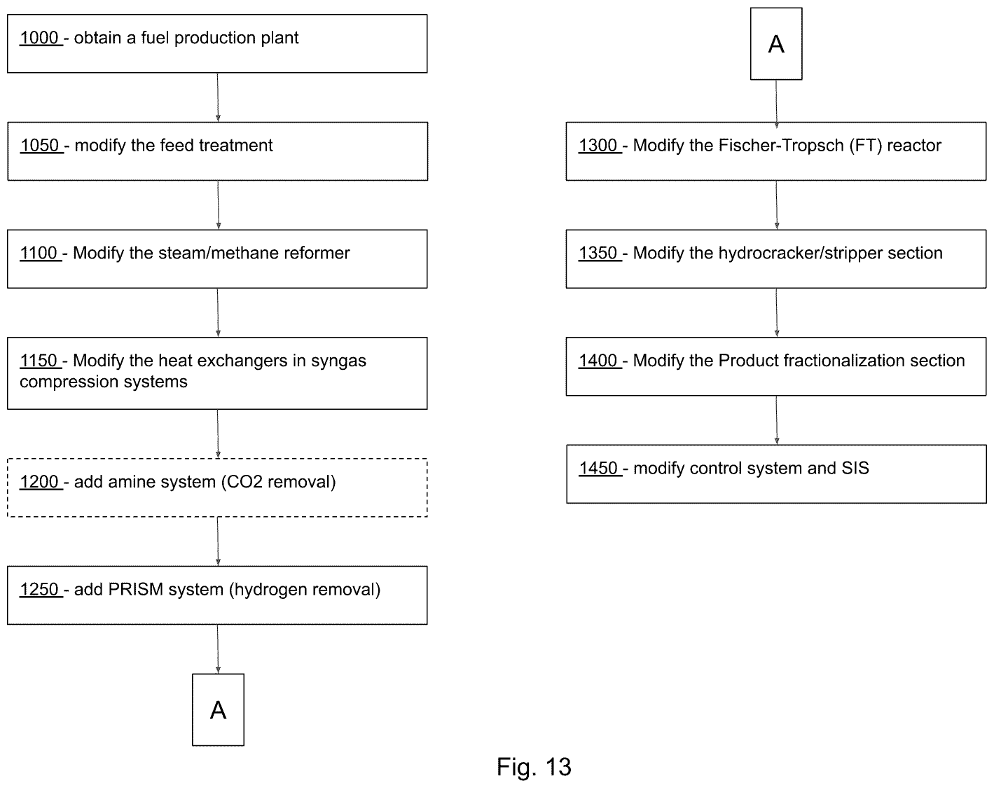

FIG. 1 depicts a converted brownfield plant, designated as 10, which has been modified according to the steps of the present invention. The brownfield plant 10 includes multiple sections or systems, each serving a specific function in the overall process. These sections are: a feed treatment section 100, a steam methane reformer section 200, a syngas compression section 300, a carbon dioxide removal section 400, a hydrogen removal section 500, a Fischer-Tropsch (FT) reactor section 600, a hydrocracker/stripper section 700, a product fractionalization section 800, and a control system 900. Each of these sections/systems is further described in reference to FIG. 1-10. Additionally, FIGS. 11 and 12 depict components of the FT reactor section 600 and FIG. 13 outlines a method for retrofitting a mothballed brownfield plant to achieve the objectives set forth in this invention.

In one embodiment, the brownfield plant 10 is initially either a mothballed or under-performing facility designed to process feedstock into liquid energy sources. The types of facilities that can be converted into brownfield plant 10 include, but are not limited to, methanol production facilities and ammonia production facilities.

The feedstocks that can be processed by brownfield plant 10 are diverse and include natural sources such as natural gas, as well as biogenic sources like biomass, renewable natural gas, hydrogen, municipal waste, and flared or stranded gas. The liquid energy sources that can be produced by brownfield plant 10 include, for example, jet fuel, LPG Gas, diesel and NAPTHA. Given its ability to process multiple types of feedstock and produce various liquid energy sources, brownfield plant 10 operates as a feedstock-agnostic facility, also referred to as an XTL plant.

### Feed Treatment 100

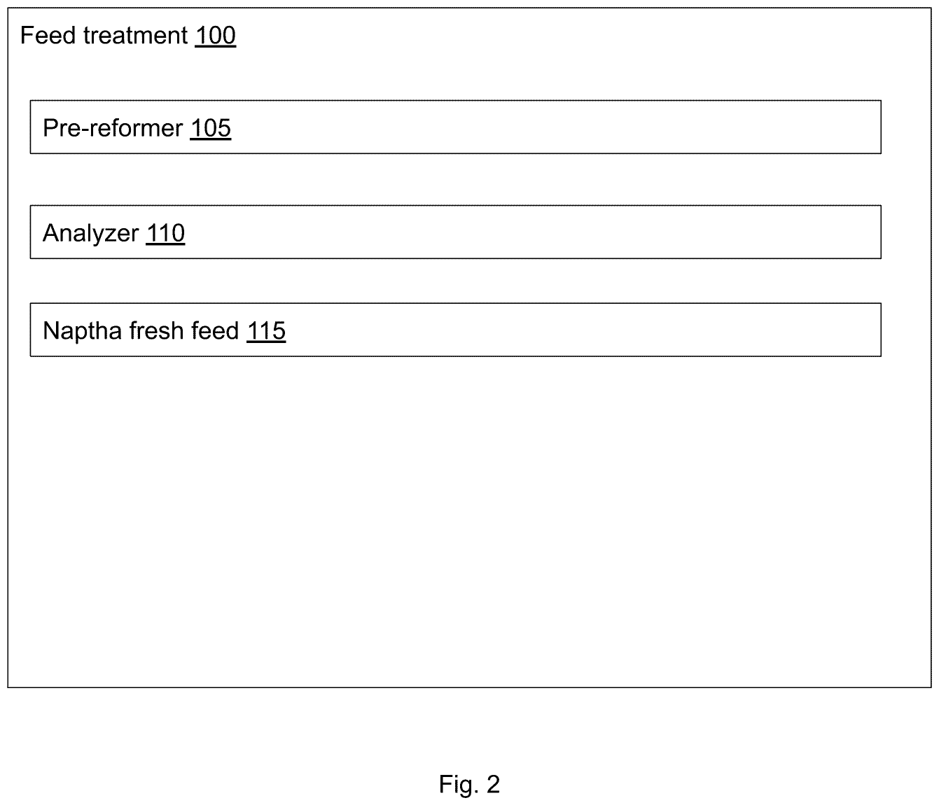

FIG. 2 illustrates the feed treatment section, designated as 100, which serves the function of pre-treating or cleaning the gasses produced from various feedstocks. Specifically, the feed treatment section 100 is configured to remove organic and inorganic sulfides from the feed gas. To achieve this, the feed treatment section 100 may include at least one reactor and one superheater. Modifications to this section may involve the addition of a prereformer 105, analyzer 110, and naphtha fresh feed 115. In an embodiment of the invention, an outlet of the feed treatment 100 may connect to an inlet of the steam methane reformer. 200.

In one embodiment related to retrofitting a brownfield plant, a pre-reformer 105 is added to the feed treatment section 100. The pre-reformer 105 is designed to break down heavy hydrocarbons in the feed gas before it proceeds to the steam methane reformer 200. Specifically, the pre-reformer 105 breaks down ethane, butane, and propane which could be present in the incoming gas stream, leaving primarily methane to be fed to the steam methane reformer. In cases where the pre-reformer 105 is not included, the steam methane reformer 200 may experience an overload due to the presence of heavy hydrocarbons, however, this can be managed by adjusting the flow feed rates to the unit.

The analyzer 110 is integrated into the system to monitor the gas composition at the output stage. The analyzer 110 may include at least one sensor capable of detecting levels of sulfur and observing the purity of the gas. Alternative types of analyzers could include different sensor technologies capable of similar measurements.

Naphtha fresh feed pump 115 may be used to redirect a slip stream of naphtha gas from the storage tank to the feed treatment section 100. When naphtha reaches the reformer and breaks down or atomizes, it may increase the charge of the reformer. This then helps in reducing the gas consumption to the facility.

### Steam Methane Reformer 200

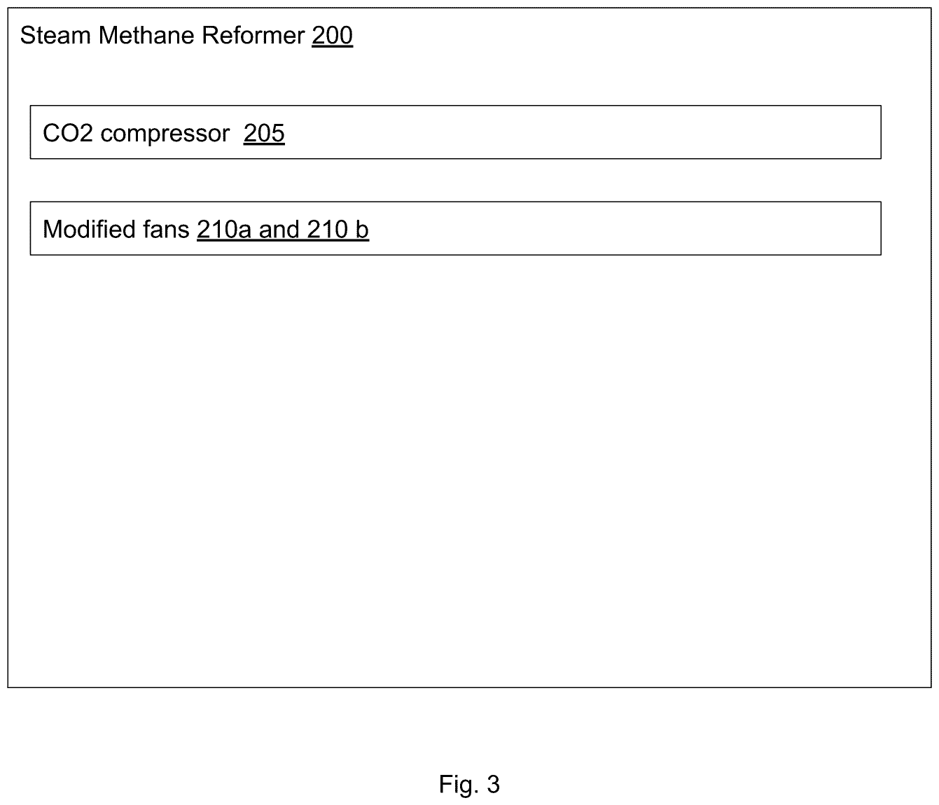

Referring to FIG. 3, the steam methane reformer section, designated as 200, serves the function of breaking down the incoming feed gas into its constituent components, namely carbon monoxide (CO), carbon dioxide (CO2), and hydrogen (H2). To facilitate this chemical transformation, the steam methane reformer section 200 may include various components such as heat exchangers, a tubular reformer (radiant box), a plurality of reformer fans, a flue stack, and steam superheater convection coils. Heat capture elements may be added to the flue stack in refitting. These steam coils are used to generate steam to drive turbines to produce energy for the plant 10. Alternatively, if the heat transfer coils were not used, electricity would need to be generated and provided to the plant through other means known in the art. A CO2 compressor 205 and modified fans 210a and 210b may be added to this system. In an embodiment of the invention, an outlet of the steam methane reformer 200 may connect to an inlet of the syngas compression and heat recovery system 300.

In operation, the feed gas entering the steam methane reformer section 200 undergoes a series of chemical reactions facilitated by the aforementioned components. The heat exchanger preheats the feed gas before it enters the tubular reformer, where the primary reforming reactions occur. The flue stack serves as an outlet for combustion gasses from the furnace, while the steam superheater elevates the temperature of steam used in the reforming process, along with other steam generated via the excess heat from the flue gas. The second heat exchanger may be used for heat recovery or other thermal management purposes.

The gas exiting the steam methane reformer section 200 is characterized by its elevated temperature and lower pressure conditions. Specifically, the gas may be heated up to a temperature of 1600 degrees Fahrenheit and may be under a pressure of 180 psi. Additionally, the gas leaving this section is in a wet state, indicating the presence of moisture content, which will be subject to further treatment for utilization in subsequent sections of the plant

Another modification involves the addition or conversion of a CO2 compressor, designated as 205. In one embodiment, the CO2 compressor 205 serves one main function. It transfers CO2 from the Amine system to the inlet of the reformer 200. This adjustment in the feed gas composition is aimed at facilitating the chemical reactions that yield higher quantities of Carbon Monoxide (CO) and Hydrogen, which are essential for the Fischer-Tropsch (FT) reactors. This re-injection of CO2 into the front end of the reformer allows for the water shift reaction to maintain a higher percentage of CO in its gaseous form, which is favorable for the FT reactor.

For brownfield plants that do not have an existing CO2 compressor, the method includes retrofitting an available natural gas compressor to serve as the CO2 compressor 205. This is particularly useful for plants that have a natural gas compressor initially intended for compressing incoming gas to higher pressures but is not required for that purpose. By converting the existing natural gas compressor into a CO2 compressor, the plant eliminates the need for additional capital expenditure on a new compressor.

A modified set of fans 210a and 210b may be used to ensure efficiency within the steam methane reformer 200. The modified steam methane reformer 200 will be processing a higher flow rate than the original brownfield plant 10. Forced draft fan 210a will be used to introduce more air into the steam methane reformer 200. Induced draft fan 210b will be used to pull more air into the flue gas to hold a negative pressure in the furnace. In an embodiment of the invention this modification results in a twenty five percent increase in capacity.

### Syngas Compression & Heat Recovery System 300

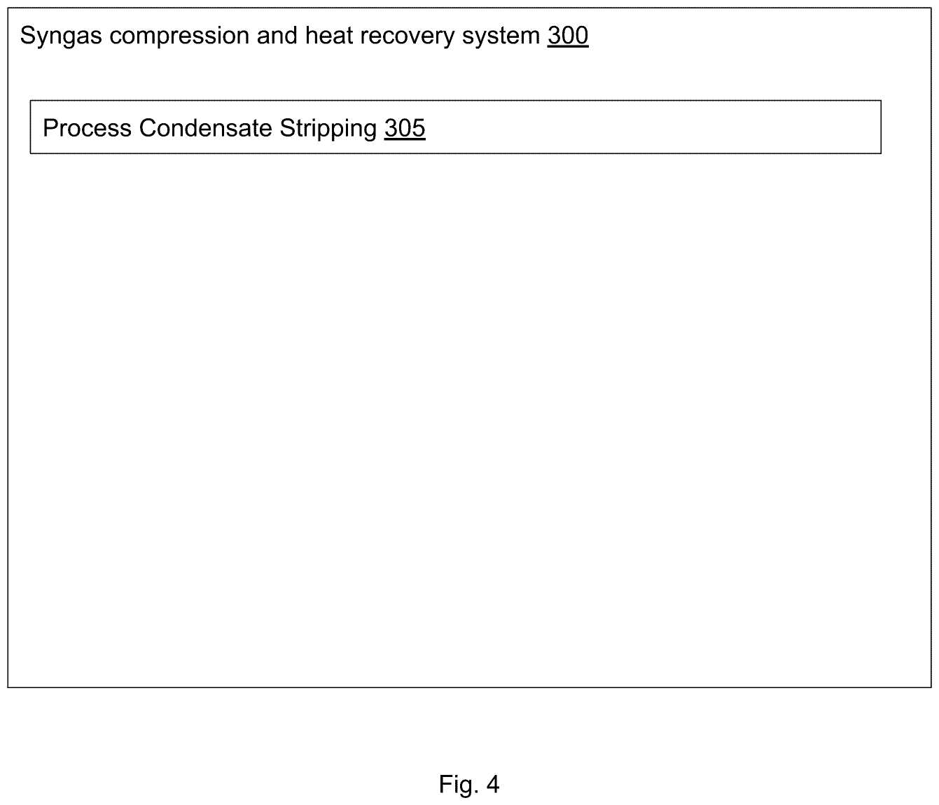

Referring to FIG. 4, the syngas compression section 300 serves multiple functions, including the conversion of wet reform gas from the steam methane reformer section 200 into dry synthesis gas. Additionally, this section captures heat for utilization in other areas of the brownfield plant 10. To achieve these functions, the syngas compression section 300 may include a high-pressure separator, one or more heat exchangers, components for process condensate stripping 305, and a compressor. In an embodiment of the invention, an outlet of the syngas compression system 300 may connect to an inlet of the carbon dioxide removal section 400

The wet reform gas initially passes through several high-pressure separators, which separates the gas from any entrained liquids. The gas then moves through a heat exchanger, which serves to both dry the gas and capture heat. This captured heat is repurposed for preheating other gas streams or boiler feed water streams, thereby optimizing energy use within the plant. The ability to cool down the gas stream and knockout the liquids occurs several times until the gas is cooled to ambient. The section may require modifications, such as the addition or alteration of existing heat exchangers in the brownfield plant 10, to perform these functions effectively.

Upon exiting the heat exchanger, the gas is directed to a compressor, which may be the largest compressor within the facility and is specifically designed for handling synthesis gas. The compressor increases the pressure of the gas from 180 PSI to 950 PSI. This high pressure is necessary for the subsequent sections of the plant, including the amine system, the PRISM unit, and the Fischer-Tropsch (FT) reactor, all of which operate more effectively at higher pressures.

Process condensate stripping (PCS) section 305 may be used to remove water from the gasses before entering the FT reactor 600 which may help reduce hazardous air pollutants. The PCS section 305 may comprise a column, and associated condensers, used to strip, with air or steam water produced in the FT reactor 600. This process may strip out the carbonic acid out of the condensate and return the pH of the condensate back to 7. In an embodiment of the invention this may save 25 to 30 percent of the water consumed by the conversion process.

The term “synthesis gas” is used to describe the gas at this stage, distinguishing it from the “reform gas” that initially entered the section. The reform gas is considered “wet” due to the presence of entrained liquids. However, after passing through the high-pressure separator and heat exchanger, these liquids are removed, and the gas is referred to as “dry” synthesis gas. The primary components of this dry synthesis gas are carbon monoxide (CO), hydrogen (H2), and carbon dioxide (CO2).

CO2 Removal System 400

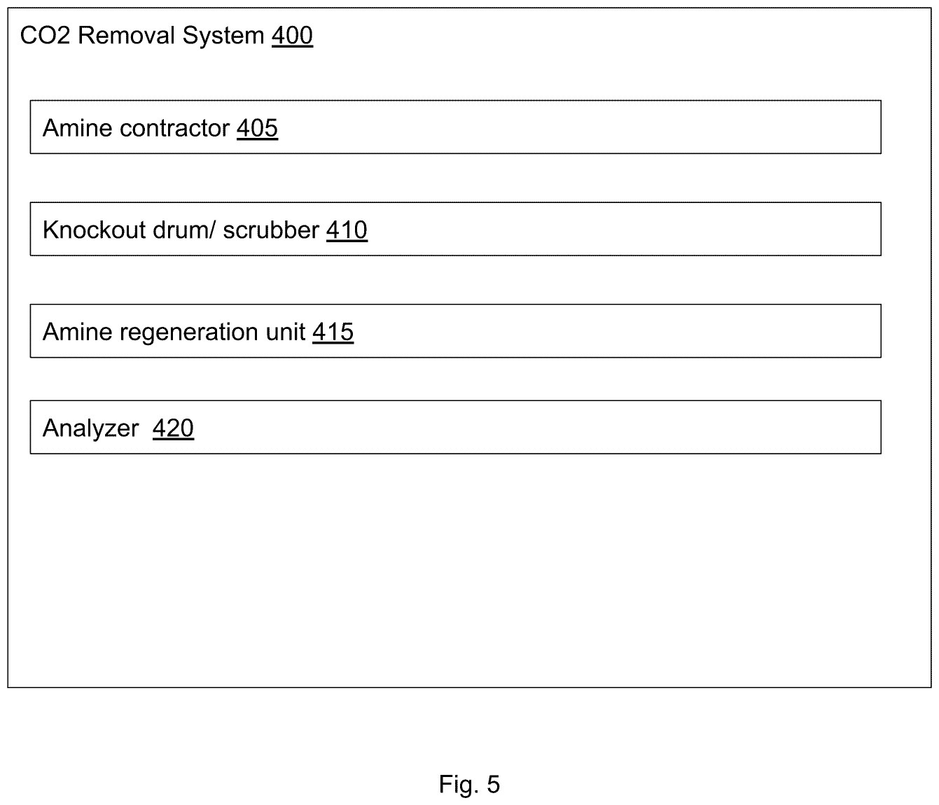

Referring to FIG. 5, the carbon dioxide removal section 400 serves to remove CO2 and H2S, commonly referred to as “Acid Gases,” from the natural gas feed. This is achieved through chemical reactions in the contactor tower 405, also known as the absorber. The tower utilizes a water solution of a weak base, specifically an alkanolamine, to form soluble salts such as amine carbamate or amine hydrosulfide. The CO2 removed is then directed to the CO2 compressor 205 for further processing. The gas exiting this section is primarily composed of hydrogen and carbon monoxide. In an embodiment of the invention, an outlet of the carbon dioxide removal section 400 may connect to an inlet of the hydrogen removal system 500.

To facilitate these processes, the carbon dioxide removal section 400 may include various components such as a separator, scrubber cooler, contractor scrubber pump, scrubber surge tank. An amine contractor 405, knockout drum/scrubber 410, and Amine regeneration unit 415, and analyzer 420 may be added to or modified in the system.

The amine contactor 405 serves to absorb CO2 and H2S from the natural gas streams. The contactor 405 may contain Monoethanolamine (MEA), Methyldiethanolamine (MDEA), or Diethanolamine (DEA) as the absorbing agent. The design of the amine contactor 405 can vary, utilizing either trays or packing material to achieve the desired separation efficiency. Mist eliminators may also be incorporated to minimize amine solvent loss.

The knockout drum/scrubber 410 may remove liquid droplets and impurities from the gas stream. These droplets may be water, hydrocarbons, or other contaminants that can affect the downstream equipment's performance or the purity of the methane gas. The drum uses gravity to separate the liquids from the gas stream, which then exits the drum through a vapor outlet The knockout drum/scrubber 410 may comprise a chemical solvent, such as an aqueous amine solution, which selectively captures CO2 molecules from the gas stream

The Amine regeneration unit 415 serves to regenerate the amine solution by stripping out the absorbed CO2 and returning the amine back into the system, thus creating a closed-loop operation.

The analyzer 420 may measure the CO2 slippage at the outlet of the CO2 removal system 400. The analyzer 420 may be a gas sensor of appropriate sensitivity as known by those skilled in the art. This measurement is the percentage of CO2 not removed. In an embodiment of the invention this level is 0.6 mol by percentage.

### Hydrogen Removal Section 500

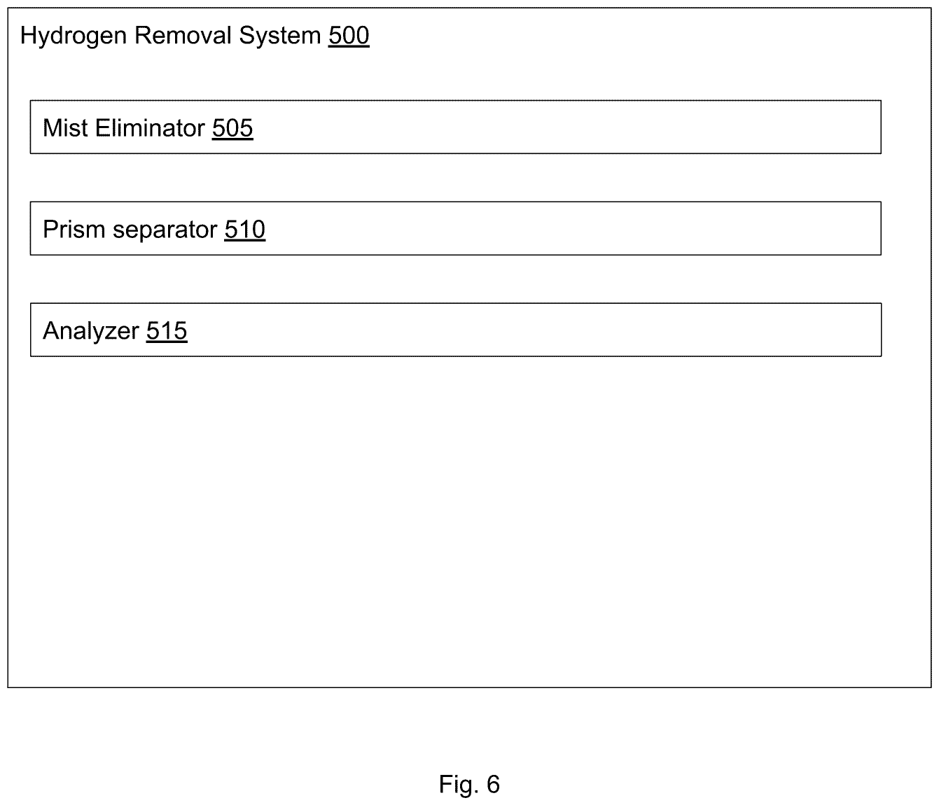

Referring to FIG. 6, the hydrogen removal system 500 serves the purpose of adjusting the hydrogen content in the synthetic gas stream. In one embodiment, the system aims to achieve a H2:CO ratio of 2.03, a ratio that is particularly relevant for GTL plants for optimal conversion of feed syngas into Fischer-Tropsch (FT) wax or syncrude. To facilitate this, the hydrogen removal section 500 may include various components such as a mist eliminator 505, a feed heater, and PRISM separators 510. Additionally, the system may be modified to include analyzer 515 for monitoring gas composition at different stages. In an embodiment of the invention, an outlet of the hydrogen removal system 500 may connect to an inlet of the Fischer-Tropsch (FT) reactor section 600.

The mist eliminator 505 is designed to remove liquid particulates from the gas feed, ensuring that the synthetic gas remains dry. This is critical in extending the life of the PRISM membranes. In the present invention, the mist eliminator 505 may consist of a filter and a vessel. The gas passes over the filter, which captures liquid particulates, allowing only dry gas to proceed. Alternative designs for the mist eliminator 505 could include different types of filters or separation mechanisms.

The PRISM separators 510 serve to remove hydrogen from the synthetic gas. In this embodiment, the PRISM separators 510 may consist of thousands of hollow fibers that function as molecular filters. These fibers separate the compressed gasses into their individual elements, effectively removing some hydrogen from the mixture. Alternative mechanisms for hydrogen separation could include other types of molecular sieves or membrane technologies. The hydrogen removed in this system is reused as a source of fuel for heaters and boilers and also fed to the Hydrocracker Section to assist in breakdown of heavy wax.

The analyzer 515 is integrated into the system to monitor the gas composition at the output stage. The analyzer 515 may include at least one sensor capable of detecting levels of H2, CO and observe the ratio to each other. Alternative types of analyzers could include different sensor technologies capable of similar measurements.

### FT Reactor 600

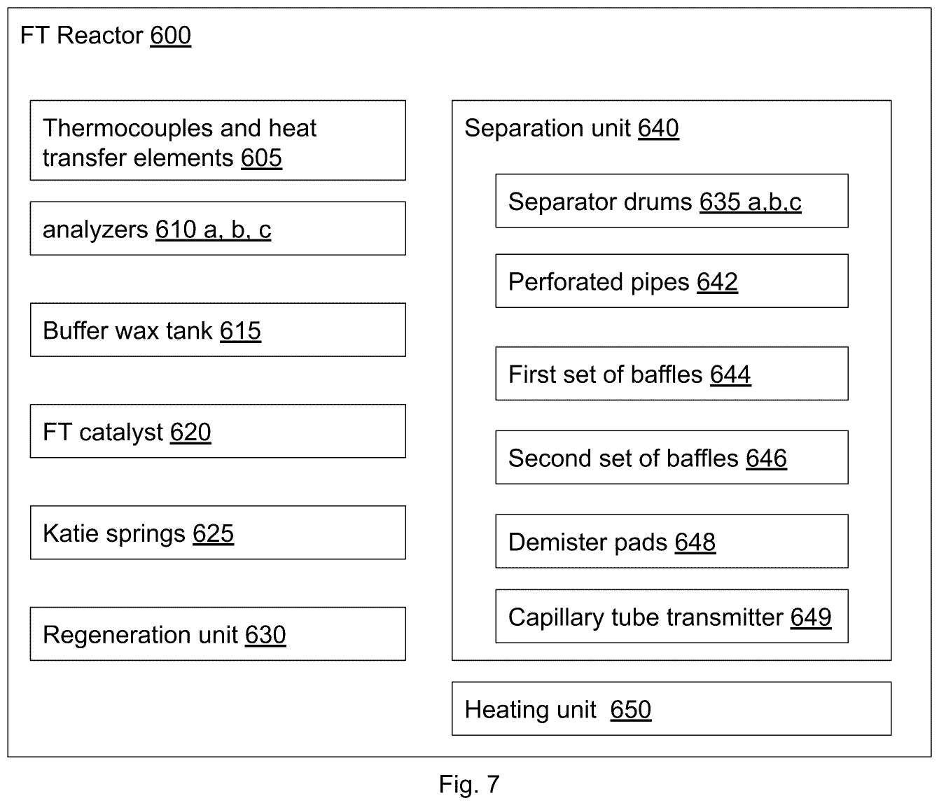

Referring to FIG. 7, the Fischer-Tropsch (FT) reactor section 600 serves to convert gaseous feed into GTL wax. The FT reactor section 600 may incorporate fixed bed technology, and may require several modifications, including the addition of thermocouples 605, heat transfer elements, analyzers 610a, b, c, a GTL buffer wax tank 615, a FT catalyst 620, Katie springs 625, a catalyst regeneration unit 630, separator drums 635a, b, c and process condensate stripping section 640. Alternative bed designs could utilize a slurry bed. In an embodiment of the invention, an outlet of the FT reactor 600 may connect to at least one of an inlet of the hydrocracker/stripper section 700 and the fractionation system 800.

The thermocouples and heat transfer elements 605 are designed for stable heat removal and temperature measurement in the reactor tubes. The present invention deploys over 250 thermocouples per reactor 600, compared to a standard of approximately 35. The heat transfer elements 605 in the present invention, may comprise a plurality of prongs, 3 or 4 in an embodiment of the invention, that form a set of walls that meet in the middle of a pipe and contact the outer walls of the pipe, subdividing the circular cross section into a plurality of lumen. Fewer prongs may be used for smaller diameter pipes and more prongs may be used for larger diameter pipes. Alternative designs could include fewer or different types of heat transfer elements.

In one embodiment, the various analyzers are strategically positioned at various points in the process flow, specifically at the inlets, outlets, and recirculation streams of the Fischer-Tropsch (FT) reactor 600. These analyzers are designed to continuously monitor the composition of the gas streams, focusing on the ratio of hydrogen to carbon monoxide. The analyzers are capable of providing real-time data, which is used for process adjustments.

In one embodiment, an inlet analyzer 610a may be placed at the inlet of the reactor 600. The inlet analyzer 610a measures the composition of the fresh feed gas being injected into the system. This allows for immediate adjustments to maintain the desired ratio of hydrogen to carbon monoxide. The primary function of the inlet analyzers 610a is to ensure that the feed gas maintains a specific molar ratio of two moles of hydrogen to one mole of carbon monoxide. The data collected by these analyzers is sent to the control system 900 via the analyzer and/or compressor interface 940, which can adjust the composition of the feed gas if deviations from the desired ratio are detected.

In another embodiment, an outlet analyzer 610b may be placed at the outlet of the reactor 600 to measure the composition of the reacted gasses, providing information on the level of conversion achieved. More specifically, outlet analyzers 610b are located at the exit point of the FT reactor 600 where reacted gasses are released. These analyzers 610b measure the composition of the exiting gas stream to determine the level of conversion that has been achieved. Specifically, the outlet analyzers 610b assess the remaining concentrations of hydrogen and carbon monoxide to gauge the efficiency of the reaction. The data from the outlet analyzers 610b is used to adjust operational parameters, such as flow rates or temperature, to optimize conversion in subsequent cycles.

A third analyzer 610c is positioned where the recycled stream, consisting of unreacted gasses, is reintroduced into the inlet of the FT reactor 600. This is relevant because this point is where the fresh feed gas mixes with the recycled stream. Continuous monitoring at this point ensures that the mixed stream maintains the desired compositional ratio for optimal conversion. More specifically, recirculation stream analyzers 610c are situated at the point where unreacted gasses are reintroduced into the inlet of the FT reactor 600. These analyzers 610c monitor the composition of the recycled gas stream, focusing on the concentrations of hydrogen and carbon monoxide. The control system 900, via the analyzer and/or compressor interface 940 uses this data to adjust the composition of the mixed stream, ensuring that it aligns with the desired molar ratio for optimal conversion.

The buffer wax tank 615 is used to maintain the wax in a molten state. The wax is kept above 212 Fahrenheit in order to prevent solidification and potential obstructions and damage to the system. The buffer wax tank 615 in the present invention, may comprise a vessel capable of holding wax in motion and a steam coil to keep the wax molten. Alternatives to the buffer wax tank 615 include but are not limited to vessels that utilize other heating methods to maintain the wax in a molten state.

The FT catalyst 620 is used to convert feed gas into FT wax or syncrude. In this embodiment, the catalyst may be held within a tube in a fixed bed. In another embodiment, the FT catalyst 620 may be a TI-8 catalyst. Alternative catalyst designs could include different types of catalytic materials or structures.

The Katie springs 625 hold the catalyst 620 within the tubes during operation. These springs may be shaped to hold the catalyst at the tube's bottom and allow case of removal during catalysts change out every 4-5 years. Alternative designs could include different types of holding mechanisms.

The regeneration unit 630 is designed to extend the life of the catalyst 620. It may consist of stacked reactors with a moving bed of catalyst. The regenerated catalyst is re-injected at the top of the first reactor, completing the circulation cycle. In one embodiment, The regeneration unit 630 for the catalyst 620 is used to bring the catalyst 620 back to the beginning of its life expectancy. Doing this helps maintain the highest productivity of the catalyst and maintain a high reactivity. The regeneration unit 630 for the catalyst 620 in the present invention, may comprise a set of tubes to hold the catalyst 620 in a fixed position. Flow may then be introduced through the tubes that are rich in hydrogen to contact the catalyst 620. The feed is then raised to the reaction temperature in the charge heater and sent to the first reactor section. Because the predominant reforming reactions are endothermic, an inter-reactor heater (loop interchanger) is used to reheat the charge to the desired reaction temperature before it is introduced to the next reactor. The effluent from the reactor is heat exchanged with the combined feed, cooled, and separated into vapor and liquid products in a separator. Alternative designs could include different types of regeneration mechanisms known to those skilled in the art. The regeneration unit may require 99.9% purity of H2. This level of purity is produced through the use of a PSA (Pressure Swing Adsorber) and the H2 drawn off the feed by the hydrogen removal section 500. The regeneration unit 630 may utilize the PSA to produce pure hydrogen as an end product when not needed for regeneration.

The separator drums 635a, b, c are used to separate the wax into different grades, heavy, middle, and light waxes. First stage hot product separator 635a separates heavy wax from the other versions of wax. Hot product liquid separator 635b may separate middle wax. Cold product separator 635c may separate light wax from the feed. The separator drums 635a, b, c in the present invention, may comprise a series of perforated pipes. Alternatives to the separator drums 63a, b, c include but are limited to fewer separator drums to be used. Alternative designs could include a different number or types of separator drums.

In one embodiment, the separation unit 640 is comprised of perforated pipes 642, first set of baffles in a first drum 644, second set of baffles in a second drum 646, and demister pads 648. Adding perforated pipes 642 to the fractionalizing unit or the separation unit 640 enables the functionality of slowing down the flow rate of the incoming mixed stream, thereby providing the necessary time for improved separation to occur downstream at the baffle plates 644, 646 and/or the demister pads 648. In one embodiment, this is achieved through the custom design of the perforations, which are calibrated to create a specific flow resistance. The perforations act as flow restrictors, generating a pressure drop that results in a laminar flow condition as the mixed stream exits the pipe and enters the vessel. In other embodiments, the perforated pipe may include a series of strategically placed perforations along its length and circumference. These perforations are engineered with specific diameters and spacing intervals to ensure an even distribution of the flow. In one embodiment, the pipe is fabricated from corrosion-resistant materials suitable for high-pressure and high-temperature environments, thereby ensuring its durability and long-term performance. By incorporating these specific design features and functionalities, the perforated pipe in the present invention offers a robust, efficient, and highly effective solution for the initial distribution and flow control in the complex task of separating different grades of GTL wax and water. This component is especially critical for brownfield plants retrofitted for fuel production, where precise control and efficient separation are paramount.

Adding a first set of baffle plates 644 in a first drum enables the retrofitted brownfield separation unit to separate heavy wax from the mixed stream. The first set of baffle plates 644 may be specifically engineered to facilitate the separation of heavy GTL wax from the mixed flow of light and medium waxes and water. In accordance with various embodiments, the first set of baffle plates 644 may be strategically positioned within the first drum following the perforated pipe 642 and may be custom-designed with particular geometries, including specific angles, dimensions, and surface treatments, to maximize the efficiency of heavy wax separation. In one embodiment, the first set of baffle plates 644 induce laminar flow conditions within the first drum, thereby allowing the heavy wax to float and separate effectively from the other components based on density differentials. The baffle plates 644 achieve this by creating zones of reduced flow velocity, which in turn provides the heavy wax with the necessary residence time to separate and float above the water and lighter waxes. Once the heavy wax reaches a predetermined level, it flows over the baffle plate 644 and is channeled for further processing, such as hydrocracking. This precise control over the separation process is crucial for directing the heavy wax to its intended downstream processing unit, thereby improving the overall efficiency and operational integrity of the plant. In one embodiment, the plates 644 are constructed from materials that are both corrosion-resistant and capable of withstanding the high-pressure and high-temperature conditions commonly encountered in brownfield plants retrofitted for fuel production.

The second set of baffle plates 646 may be comprised of retrofitting an existing knuckle drum in a separation unit or installing a new one either of which is hereinafter also referred to as a second drum. In the second drum, a similar process occurs to further separate middle and light waxes. In some embodiments, the second set of baffle plates 646 may even be installed in the first drum. In accordance with various embodiments, the second set of baffle plates 646 may be designed with specific geometries, including angles and dimensions, to facilitate the separation of medium and light GTL waxes from the remaining mixed flow. The materials selected for the construction of these baffle plates are resistant to corrosion and capable of withstanding high-pressure and high-temperature conditions. The function of the second set of baffle plates 646 is to create zones of reduced flow velocity within the second drum. This reduced flow velocity provides the medium and light waxes with the residence time needed to separate based on their densities. Once the separation occurs, the medium and light waxes are directed to their respective downstream processing units, such as fractionation columns. The second set of baffle plates 646 work in conjunction with the first set of baffle plates 644 in the first drum, offering a two-stage separation process that enhances the overall performance of the separation unit. By incorporating these design features, the second set of baffle plates 646 in the second drum provides an effective mechanism for the separation of medium and light GTL waxes. This component is used in brownfield plants retrofitted for fuel production, where accurate separation and channeling of material streams are necessary for plant operation.

In one embodiment, the demister pads, 648 which may be positioned at the end of the separation unit 640, may further aid in the separation process by capturing any remaining droplets of liquid in the gas phase. In one embodiment, the demister pads 648 are installed in the second drum and/or outside the second drum to ensure that any entrained gas is also separated from the liquid waxes. In one embodiment, the demister pads 648 are constructed from a mesh-like material designed to capture liquid droplets from the gas phase. The mesh is selected based on its ability to withstand the chemical composition of the mixed flow, as well as high-pressure and high-temperature conditions commonly encountered in the separation process. The dimensions and density of the mesh are engineered to maximize the surface area for droplet capture while minimizing flow resistance. In operation, the demister pads 648 serve to capture any remaining liquid droplets that may be present in the gas phase after the separation process facilitated by the baffle plates 644, 646. The pads 648 effectively increase the purity of the separated gas by removing these droplets. The liquid captured by the demister pads 648 is then drained and channeled to appropriate downstream processing units. The demister pads 648 are designed to work in conjunction with the perforated pipe 642 and the sets of baffle plates 644, 646 in the first and second drums, contributing to a comprehensive separation process.

In one embodiment, the capillary tube transmitters 649 are employed for measuring level, pressure, and temperature within the separation unit 640. In one embodiment, these transmitters 649 feature a diaphragm mechanism and are connected to the separation unit 640 via capillary tubes. The tubes 649 are filled with a fluid that transmits pressure changes to the diaphragm, which in turn generates an electrical signal corresponding to the measured parameter. The materials used for the construction of the capillary tubes 649 and the diaphragm are selected for their chemical resistance and suitability for high-pressure and high-temperature conditions. In operation, the capillary tube transmitters 649 provide measurements without allowing the medium-whether it be wax or water-to come into direct contact with the sensors. This design prevents clogging and ensures the accuracy of the measurements. The electrical signals generated by the diaphragm are then converted into readings for level, pressure, and temperature, which are used for process control and monitoring. These transmitters 649 are integrated into the overall control system of the separation unit 640 and work in conjunction with the perforated pipe 642, baffle plates 644, 646, and demister pads 648 to facilitate the separation process.

The heat transfer elements 650 are used to maintain the wax temperature above 212 degrees Fahrenheit. The heat transfer elements 650 may comprise additional steam tracing that has been added to the brownfield plant 10 to capture heat losses or waste from other sections and direct it back to the hydrocracker/stripper section 700. Alternative designs could include different types of heat maintenance systems.

### Hydrocracker/Stripper Systems 700

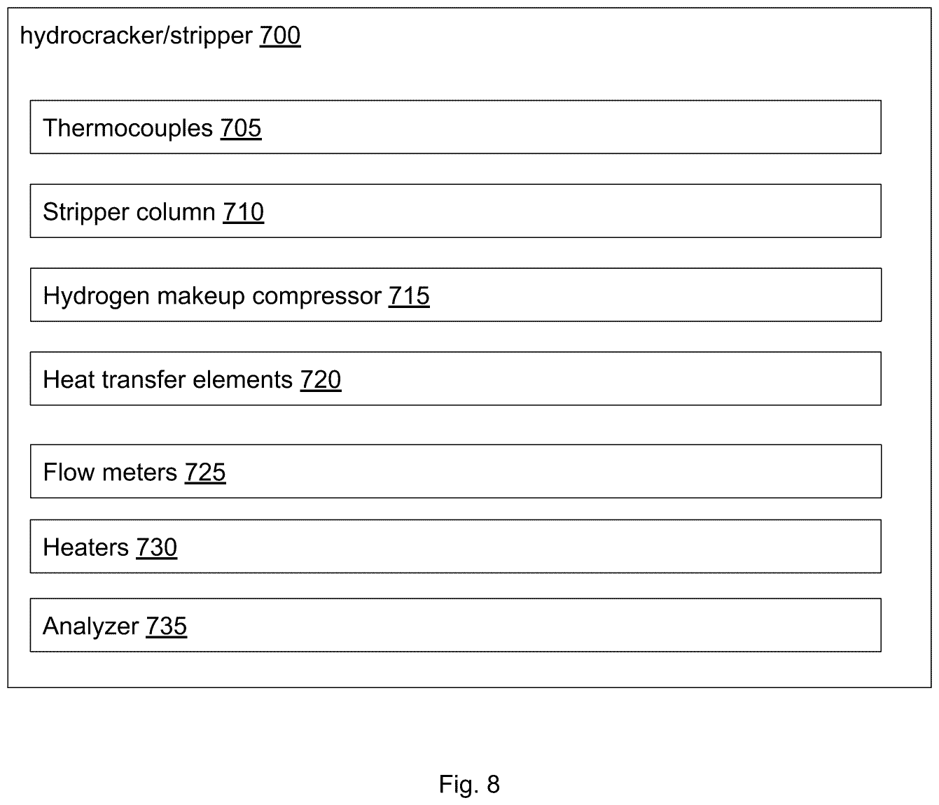

Referring to FIG. 8, the hydrocracker/stripper section 700 serves multiple functions within the brownfield plant 10. This section may include a stripper reflux drum, stripper reflux pump, reactor, HCU heater, flash drum, effluent feed, strippers, and a stripper feed preheater. Modifications to this section may involve adding thermocouples 705, a stripper column 710, a hydrogen makeup and recycle compressor 715, heat transfer elements 720, flow meters 725, heaters 730, and analyzer 735. In an embodiment of the invention, an outlet of the hydrocracker/stripper section 700 may connect to an inlet of the fractionation system 800.

The thermocouples 705 are designed for measurement of temperature through multiple levels of the hydrocracking reactor column 700. In an embodiment of the invention, the thermocouples 705 are located in at least 8 levels of the column to measure the temperature of the catalyst in each level. This additional measurement and control capability allows the control system 900 to better adjust for changes in the system to maintain efficiency.

The stripper column 710 is used to separate components within the feed using a vapor stream. Specifically, it may break down heavy wax into medium and light versions. The stripper column 710 in the present invention, may comprise a column that is capable of holding the heavy wax in a liquid state and capable of passing vapor through the column. Alternative designs could include different types of separation mechanisms.

The hydrogen makeup compressor 715 introduces H2 into the hydrocracker/stripper section 700. It may be comprised of a compressor that draws H2 from the Hydrogen removal section 500. Alternative designs could include different types of hydrogen sourcing mechanisms.

Heat transfer elements 720 and heaters 730 are added to elevate the temperature of wax entering the hydrocracking section 700. The heat transfer elements 720 may comprise boilers or other heating elements known in the art. The heaters may comprise steam tracing heaters or other heat sources known in the art.

The flow meters 725 are used to provide high precision flow and density measuring information about the flow of wax through the system. The flow meters may directly measure the fluid mass flow in the closed pipeline as a high precision mass flowmeter. They may use multi-variable digital processing measurement technology, the application of which strengthens the signal filtering, greatly improves the sensitivity and accuracy of signal measurement, speeds up the response time of the system, and makes the measurement more reliable. The flow meters 725 may provide a measurement accuracy of mass flow that can reach 0.1%˜0.2%. The density measurement resolution can reach 0.002˜0.02g/cm3. Also, The measurement error of temperature is less than 0.5 degrees. In an embodiment of the invention the flow meters 725 may be coriolis flow meters. These special flow meters do not get clogged with the wax being produced. The flow meters 725 eliminate vibrations and gaseous medium in piping to record strictly liquid wax flow for accuracy of measurement.

The analyzer 735 is integrated into the system 700 to monitor the gas composition at the output stage. The analyzer 735 may include at least one sensor capable of detecting levels of sulfur and observing the purity of the gas. Alternative types of analyzers could include different sensor technologies capable of similar measurements.

### Fractionation System 800

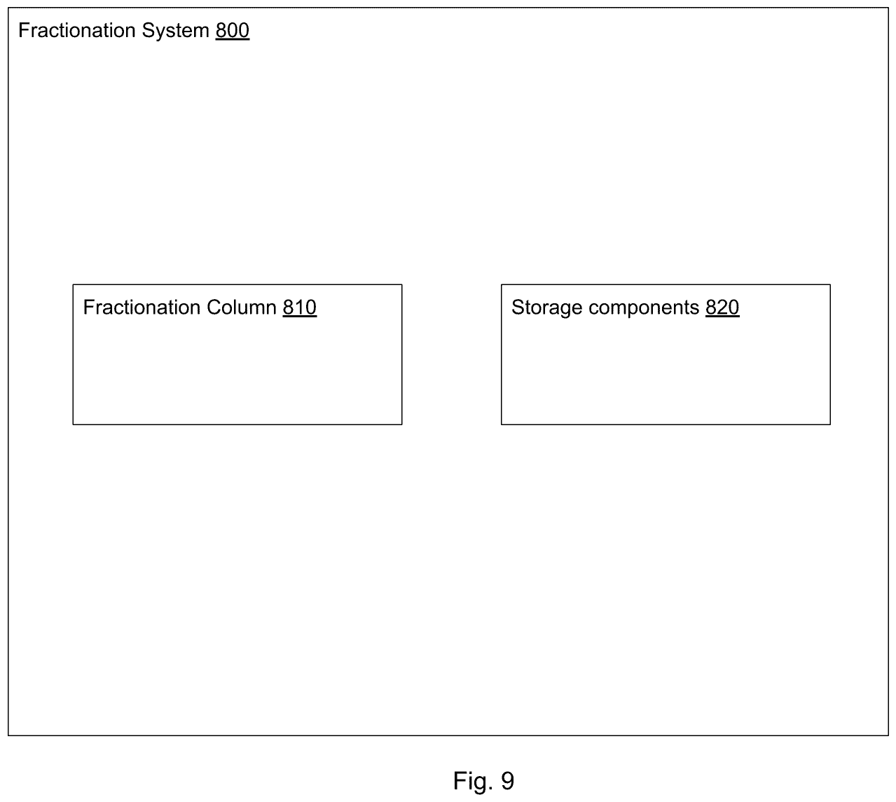

FIG. 9 illustrates the fractionation system 800. In one embodiment, it may be comprised of a fractionation column 810 and storage components 820.

In one embodiment, the fractionation column 810 comprises two main columns, distinct from those in existing methanol and ammonia plants by virtue of their unique internals and an additional vessel. The first column serves the purpose of stripping off diesel, while the second column is designed for the separation of lighter hydrocarbons. In one embodiment, the first column operates as a conventional distillation column, utilizing a temperature gradient across its trays to facilitate proper separation of components. At the bottom of this column, heavy wax accumulates. In the middle section, a draw-off point is established for diesel, and at the top, another draw-off point is designated for hydrocarbons such as naphtha and LPG. Both diesel and naphtha are then directed to smaller, secondary columns for further purification before proceeding to the final product stage. The heavy wax collected at the bottom of the first column is not discarded but is instead directed to a second reactor, herein referred to as a hydrocracker for further processing. In one embodiment, the hydrocracker cracks the wax over a second time and then eventually sends it back to the column to be purified again. This ensures that all components are efficiently utilized in the system.

In one embodiment, the storage components 820 comprise a plurality of transfer pumps, day tanks and main storage tanks. Some plants may lack these tanks initially and may need to be incorporated in the refitting process. These tanks are capable of storing and maintaining on spec the jet fuel, diesel, naphtha drilling muds, LPG and wax produced from the refitted plant 10. The tanks are piped in a way that allows for isolation and testing of the product before moving the product to a main storage tank. In another embodiment of the invention the storage components 820 may comprise fewer tanks and the final products can be piped to a third party that handles the main storage of the products.

### Control System 900

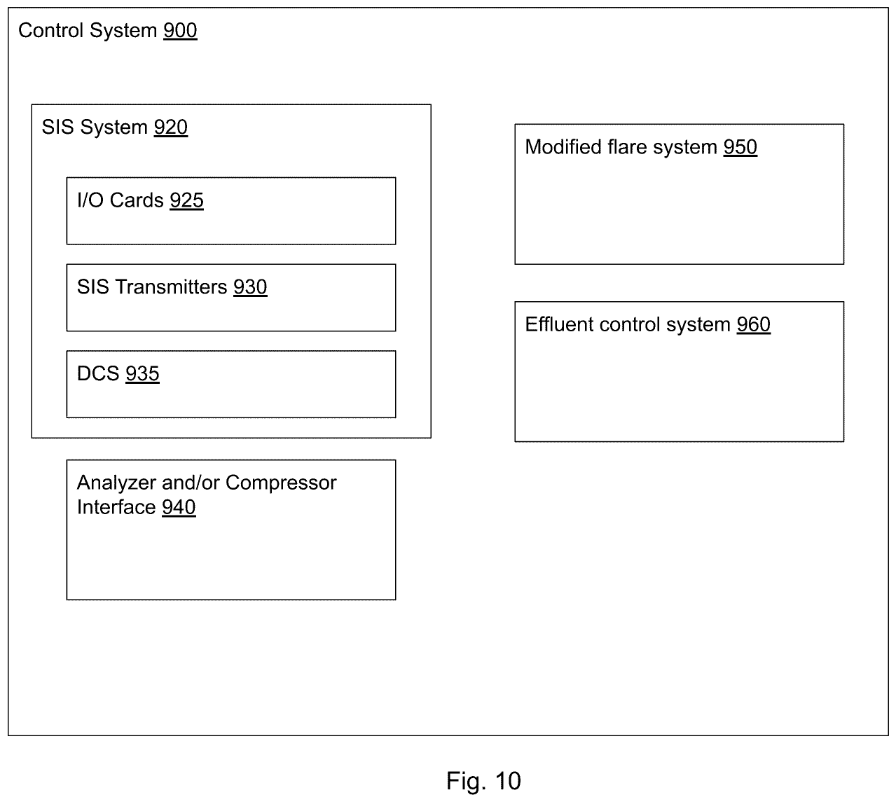

FIG. 10 illustrates an embodiment of the control system 900. The control system incorporates various analyzers and electronic components to monitor the retrofitted plant 10 and control various systems and/or subcomponents.

In one embodiment, the control system 900 is comprised of an analyzer and/or compressor interface 940. The analyzer and/or compressor compressor interface 940 is integrated with two compressors: one for circulating the gas through the reactor and another for injecting fresh feed gas. The analyzer and/or compressor interface 940 interfaces with the analyzers found throughout the plant, and works in conjunction with the compressors to ensure that the gas composition remains within the specified parameters. If the analyzers detect that some gasses did not react as expected, the control system 900 allows for the unreacted gasses to be recirculated for additional passes through the reactor. This feature enables the process to achieve maximum conversion efficiency by utilizing all available reactants.

In one embodiment, the control system 900 may be further comprised of modifying the control system and/or the SIS system 920 to improve safety and operational efficiency. As discussed above, the present invention provides a method for retrofitting a brownfield plant for fuel production by incorporating a Safety Instrumentation System (SIS). Unlike generic SIS or Emergency Shutdown Systems (ESD) used in other plants, the SIS in this invention is specifically designed and configured for the unique operational requirements of the brownfield plant being retrofitted.

In one embodiment, the SIS 920 serves as an automated control system that encompasses various protection mechanisms across the facility. It is designed to safely shut down the plant in the event of operational anomalies or emergency scenarios, without requiring manual intervention from the operator. The system can also isolate and protect specific areas or systems within the plant if it detects that something is incorrect or needs immediate attention. In one embodiment, the SIS is programmed with a set of predefined safety parameters and logic sequences that are tailored to the specific operational requirements of the brownfield plant. It continuously monitors various data points, such as temperature, pressure, and flow rates, through a network of sensors and transmitters. If any of these parameters deviate from the set safety margins, the SIS 920 triggers an automated response to either correct the deviation or safely shut down the affected system. The SIS 920 may comprise a set of cause and effect matrices for controlling the plant in case of failure.

In accordance with an embodiment of the invention, the SIS 920 may be comprised of I/O Cards (Input/Output Cards) 925. These modules are incorporated into the SIS 920 to facilitate data transfer. The I/O cards 925 are designed to work with specific types of transmitters that feed information back to the control system 900. In one embodiment of the invention, the I/O cards 925 are high-speed data acquisition cards designed to interface with the plant's existing control systems. They are equipped with multiple channels to accommodate a variety of analog and digital signals. Each I/O card 925 is encased in a flameproof housing and is certified for use in hazardous environments.

Additionally, the SIS system 920 may be comprised of specialized transmitters 930 that are used to send data to the I/O cards 925. In one embodiment, the transmitters 930 convert physical parameters like temperature and pressure into electrical signals. These signals are then sent to the I/O cards 925 via shielded cables to minimize electromagnetic interference. Each transmitter 930 is calibrated for accuracy and is capable of self-diagnostics to alert the SIS 920 in case of a malfunction.

Optionally, the SIS system 920 is further comprised of a Distributed Control System (DCS) 935, which is integrated with the SIS 920 to provide a comprehensive control solution for the plant. In one embodiment, the DCS 935 serves as the central hub for data aggregation and processing. The DCS 935 may be equipped with redundant processors and power supplies to ensure uninterrupted operation. The DCS 935 may be programmed to execute complex logic operations based on the data received from the I/O cards 925 and transmitters 930, thereby enabling real-time decision-making.

The modified flare system 950 may be used during an emergency release to recover any liquids that would have been discharged from the system to deflate. Additional knockout drums may be present in the modified flare system 950 as well as piping capable of conducting a steaming out of the flare. This process ensures that there are no deposits of product within the flare by applying 175 psi steam to steam the flare and remove deposits.

The effluent control system 960 is used to ensure that the effluent introduced to the environment from the plant 10 is within control limits. This minimizes the impact the refitted plant 10 has on the environment. The effluent control system 960 may comprise a bioreactor that comprises at least one of pH controls, bacterial controls, aeration, UV light, and filtration components. The effluent control system 960 may comprise a storm water basin. The storm water basin is capable of directing, storing and treating any surface runoff from the plant 10. Alternatively, the effluent control system 960 may include carbon capture components as well to minimize the release of CO and CO2. Any unreacted gasses produced by the plant 10 are recovered and used as fuel for heaters and furnaces.

### Liquid-Liquid Separator

FIG. 11 illustrates an embodiment of the internals of a liquid to liquid separator found within the FT reactor 600 in an embodiment of the invention. The separator in FIG. 11 may be representative of at least one of the separator drums 635a, 635b, and 635c. The separator may be comprised of a straight feed pipe 2050, a turbulence isolation plate 2100, a liquid to liquid coalescing media 2150, and a weir 2200.

### Product Separator

FIG. 12 illustrates an embodiment of the internals of a product separator found within the FT reactor 600 in an embodiment of the invention. The product separator may be comprised of a mist eliminator with liquid downcomer 3050 and a vane inlet device 3100.

### Method of Retrofitting a Brownfield Plant

FIG. 13 illustrates a method for refitting a brownfield plant 10. The steps include obtaining a fuel production plant 1000, modifying the feed treatment 1050, modifying the steam/methane reformer 1100, modifying the heat exchangers in syngas compression systems 1150, adding an amine system 1200, adding a PRISM system 1250, modifying the Fischer-Tropsch (FT) reactor 1300, modifying the hydrocracker/stripper 1350, modifying the product fractionalization section 1400, and modifying the control system and SIS 1450. Each of the above steps may be unneeded based on the makeup of the obtained brownfield plant.

Obtaining a fuel production plant 1000 may comprise steps to locate a fuel production plant for modification. The plant may utilize various feedstocks, which include, but are not limited to natural gas, biomass, renewable natural gas, hydrogen, municipal waste, and flared or stranded gas. In an embodiment of the invention the fuel production plant may be an underperforming or abandoned plant. Obtaining the plant may be accomplished by many methods known in the art and in an embodiment of the invention the plant is purchased.

Modifying the feed treatment 1050 may comprise steps to improve the efficiency of the feed treatment of the plant. A pre-reformer 105 may be added to remove ethane, butane, and propane, leaving primarily methane in the feed. The feed treatment section may also modify or add a superheater and reactor to the feed treatment section. A carbon dioxide compressor may be added to the inlet of the feed treatment portion 100 of the plant. The carbon dioxide compressor reintroduces carbon dioxide into the feed to the front end of a steam methane reformer. The carbon dioxide compressor may be refitted from an already existing natural gas compressor present in the plant before refitting. The modification of the feed treatment 1050 may include adding or modifying analyzers 110 on the feed treatment and pairing with a control system.

Modifying the steam/methane reformer 1100 may comprise steps to modify the already existing steam methane reformer present in the plant. The modifications may include at least one of adding or modifying the fans present on the flue, improving heat recovery on the flue through the addition of additional coils for heat capture. The fans may be modified by increasing airflow capability to improve efficiency within the system. The heat recovery within the steam methane reformer may be adapted to provide heat to the rest of the plant or to drive turbines for energy generation within the plant.

Modifying the heat exchangers in syngas compression systems 1150 may comprise modifying the drums within the already existing syngas compression system. The modification may result in greater amounts of water removal from the system. The drums may better capture heat from the gas passing through the section of the plant and produce dryer gas and capture heat for use in the rest of the plant.

Adding an amine system 1200 may comprise adding or modifying an already existing amine system for carbon dioxide removal. The modification may comprise adding analyzers. In some embodiments of the invention, a carbon dioxide removal system may not be present in the plant and therefore the amine system would be added as a new system instead of as a modification to an existing amine system. The amine system addition may comprise steps to add or modify a separator, scrubber cooler, contractor scrubber pump, scrubber surge tank, amine contractor, knockout drum/scrubber, amine regeneration unit, and analyzers.

Adding a PRISM system 1250 may comprise steps to add a hydrogen removal system 500. The plant may not contain a hydrogen removal system and therefore will require one to be added. The PRISM system may be used to pass gas through and remove excess hydrogen to be redirected to a hydrocracking/stripping system 700 present in the plant. The PRISM system addition may also comprise adding analyzers capable of measuring the ratio of hydrogen to carbon monoxide in the system. The PRISM system addition 1250 may also comprise steps of adding a mist eliminator to the section to reduce liquid particulates within the system. Adding the PRISM separator may comprise adding a highly efficient separator membrane, capable of producing 96 percent pure hydrogen gas.

Modifying the Fischer-Tropsch (FT) reactor 1300 may comprise steps to modify an FT reactor 600 present in the plant. The steps may comprise adding katie springs 625 to the reactor 600, analyzers 610a, b, c to the reactor 600, capillary tubes 649, heating systems 650 and adding a proper FT reactor. Modifications to an already existing FT reactor may comprise at least one of adding or replacing thermocouples and heat exchangers, modifying a buffer wax tank, changing the catalyst to a GTL wax catalyst, and modifying the separation unit 640. Adding thermocouples 605 may greatly increase the number of thermocouples within the system and improve the design of the thermocouples. Analyzers 610a, b, c may be modified or added to ensure that the gas found within the FT reactor 600 is at proper ratios and connected to a control system to maintain these ratios. A buffer wax tank may be added in a step to maintain the FTL wax within a liquid state. Modification of the FT catalyst may comprise adding a GTL wax catalyst to the system. Katie springs 625 may be added to maintain the catalyst in a proper location within the FT reactor 600. A regeneration unit 630 may be added to the plant to maintain the FT catalyst 620 in an efficient condition. Separator drums comprising a series of baffles 644, 646, demister pads 648 and capillary tube transmitters 649 may be added to separate light, medium, and heavy waxes and direct the flow to the proper location within the plant. Proper heating units 650 may be added or modified to maintain the wax in a molten state.

Modifying the hydrocracker/stripper 1350 may comprise at least one step to add a hydrogen makeup compressor 715 and heaters 730, and modify thermocouples 705, flow meters 725 and analyzers 735 already present in the plant. The step of adding the hydrogen makeup compressor 715 comprises the connection between an outlet of the PRISM system and the hydrocracker/stripper 700 to improve efficiency within the plant. Specialized flow meters 725 may be added when modifying the hydrocracker/stripper 1350 that can handle GTL wax without clogging. Modifying the hydrocracker/stripper 1350 may comprise adding heat transfer elements 720 to improve how GTL wax moves through the piping in the plant. A stripper column 710 may need to be added if not present in the plant.

Modifying the product fractionalization section 1400 may comprise at least one step of adding or modifying a fractionation column 810 and modifying the storage components 820 present in the plant. If a fractionation column is not present in the plant, it will be added in the step of modifying the product fractionalization section 1400. Modifications to the fractionation column 810 may comprise steps to improve the ability to separate GTL wax. In one embodiment, the method is comprised of modifying a separation unit. Some brownfield plants include a separation unit, but these traditional separation units are very simple because traditional methanol and/or ammonia plants only separate two streams of material (methanol and water). The retrofit methodology of the present invention updates the internals of the fractionation section or separation unit to address the unique challenges of separating different grades of Gas-to-Liquid (GTL) wax along with water in retrofitting brownfield plants for fuel production. The retrofit methodology incorporates several innovative features that improve its performance compared to traditional separation systems, including, but not limited to perforated pipes 642, a first set of baffle plates 644 in a first drum, a second set of baffle plates 646 in a second drum, demister pads 648, and/or capillary tube transmitters 649. Broadly, the updated fractionalization section or separation unit accepts mixed flow, which first enters the perforated pipe, where it is evenly distributed across the horizontal vessel. The perforated pipes 642 additionally slow down the velocity of the mixture and induce laminar flow conditions. Thereafter the mixture encounters a first set of baffle plates 644 in a first drum, which allows heavy wax to separate from other material. Thereafter, the material encounters a second set of baffle plates 646 that allows the middle wax and light wax to separate from the remaining components, with water settling at the bottom and different grades of wax floating above it. Demister pads 648 may also be installed in this secondary drum or another part of the separation unit to ensure complete separation. These updated internals separate the components to their respective streams for further processing. For example, heavy wax is directed to the hydrocracker 700, while middle and light waxes are sent to the fractionation columns 810 for purification. The separated water is used to generate steam, reducing the need for external water sources. Modification of the storage components in the plant may comprise steps to ensure the piping is properly heated to maintain the wax in a liquid state and modifying the storage tanks based on how quickly the final product may be removed from the plant.

Modifying the control system and SIS 1450 may comprise at least one step of modifying the SIS system 920, the analyzer interfaces, the flare system, and modifying the environmental controls. Modification of the environmental controls may comprise at least one of adding bacterial controls, aeration, UV light, filtration components and modifying the pH controls. Modifying the SIS system 920 may comprise creating a plurality of decision matrices for operation of the plant within normal and abnormal situations. Modification of the analyzer interfaces 940 may comprise adding additional interfaces and coupling to the additional analyzers present in the plant. Modifications to the flare system may comprise adding piping that allows for steps to steam out the flare to prevent wax buildup.

### Additional Considerations

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and

“connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A fuel production plant, comprising:
a carbon dioxide removal system, the carbon dioxide removal system comprising at least one of an amine system and an amine gas analyzer;
a steam methane reformer, the steam methane reformer comprising a carbon dioxide pump connected to at least one output from the carbon dioxide removal system;
a hydrogen removal system, the hydrogen removal system comprising a PRISM separator and at least one of a mist eliminator and a PRISM gas analyzer;
a Fischer-Tropsch (FT) reactor, the FT reactor comprising a buffer wax tank, a gtl wax catalyst, a plurality of separator drums, a FT heating unit and at least one of: a plurality of FT thermocouples, a plurality of FT analyzers, a set of katie springs, a catalyst regeneration unit, a plurality of heat transfer elements, and a plurality of capillary tubes; and
a control and safety system, the control and safety system comprising at least one of a safety instrumented system (SIS), an analyzer interface, and a flare system.

2. The fuel production plant in claim 1, wherein the fuel production plant is at least one of: an ammonia plant, a methanol plant, and a petrochemical plant in a brownfield environment.

3. The fuel production plant in claim 1, further comprising a feed treatment system for acquiring feedstock.

4. The fuel production plant in claim 3, wherein the feed treatment system comprises a feedstock gas analyzer configured for analyzing the sulfur content in the feedstock.

5. The fuel production plant in claim 3, wherein the feed treatment system comprises piping for a fresh feed of naphtha that is redirected from at least one output of the fuel production plant.

6. The fuel production plant of claim 3, wherein the feed treatment system comprises a pre-reformer.

7. The fuel production plant in claim 1, wherein the steam methane reformer further comprises an induced draft fan and a forced draft fan.

8. The fuel production plant in claim 7, wherein the steam methane reformer further comprises a flue adapted to recover heat from the gas passing through the flue and convert the heat to power turbines in the plant.

9. The fuel production plant in claim 1, further comprising a synthetic gas compression and heat recovery system, the synthetic gas compression and heat recovery system comprising a process condensate stripping system.

10. The fuel production plant in claim 1, wherein the amine gas analyzer is configured to analyze carbon dioxide slippage.

11. The fuel production plant in claim 1, wherein the PRISM gas analyzer is configured to analyze the ratio of hydrogen and carbon monoxide leaving the hydrogen removal system.

12. The fuel production plant in claim 1, wherein the PRISM separator is able to produce at least 96 percent pure hydrogen gas.

13. The fuel production plant in claim 1, wherein the buffer wax tank further comprises a plurality of steam coils.

14. The fuel production plant in claim 1, wherein the FT thermocouples comprise a plurality of prongs placed within a pipe that meet at a central axis within a pipe and extend to the interior of the walls of the pipe.

15. The fuel production plant in claim 1, wherein the FT analyzers comprise at least one of an analyzer on the input, an analyzer on the output and an analyzer on the recycle feed in the FT system.

16. The fuel production plant in claim 1, wherein the gtl wax catalyst is a FT catalyst.

17. The fuel production plant in claim 1, wherein the set of separator drums comprises a first set of baffles, a second set of baffles and a set of demister pads.

18. The fuel production plant in claim 17, wherein the set of separator drums induces laminar flow in the gtl wax produced by the FT reactor.

19. The fuel production plant in claim 1, further comprising a hydrocracker and stripper system, the hydrocracker and stripper system comprising at least one of: a hydrocracker set of thermocouples, a hydrogen makeup compressor, a hydrocracker heating unit, hydrocracker flow meters, and a hydrocracker gas analyzer.

20. The fuel production plant in claim 19, wherein the hydrocracker flow meters comprise coriolis flow meters.

21. The fuel production plant in claim 1, further comprising a fractionation system, the fractionation system comprising at least one of a fractionation column and a set of storage components.

22. The fuel production plant in claim 1, wherein the flare system comprises knockout drums and piping capable of steaming out the flare.

23. The fuel production plant in claim 1, further comprising an effluent control system, the effluent control system comprising at least one of a bioreactor and a storm water basin.

24. The fuel production plant in claim 23, wherein the bioreactor comprises at least one of pH controls, bacterial controls, aeration, UV light, and filtration components.

25. The fuel production plant in claim 1, wherein the SIS comprises a computer readable medium, the computer readable medium comprising a set of cause and effect matrices for controlling the plant in case of failure.

26. The fuel production plant in claim 1, further comprising a feed treatment system, wherein the output of the feed treatment system is connected to the steam methane reformer input;
further comprising a synthetic gas compression section, wherein the steam methane reformer output is connected to the input of the synthetic gas compression section and the output of the synthetic gas compression section is connected to the input of the carbon dioxide removal system;
wherein at least one output of the carbon dioxide removal system is connected to the hydrogen removal system;
further comprising a hydrocracker and stripper system, wherein at least one output of the hydrogen removal system is connected to the input of the FT reactor and at least one output of the hydrogen removal system is connected to the hydrocracker and stripper system; and
further comprising a fractionation system, wherein at least one output of the FT reactor and at least one output of the hydrocracker and stripper system are connected to at least one input of the fractionation system.

27. A method for refitting a brownfield plant, comprising the steps of:
obtaining a fuel production plant, wherein said fuel production plant may be an ammonia or methanol plant, or a petrochemical plant in a brownfield environment;
modifying a feed treatment system by adding at least one of a pre-reformer, an analyzer, and a naphtha fresh feed;
modifying a steam methane reformer by repurposing a CO2 compressor, improving heat capture at the flue, and modifying fans;
modifying heat exchangers in syngas compression systems;
adding or modifying an amine system for syngas compression and heat recovery;
adding a PRISM system for adjusting the ratio of H2 and CO;
modifying a Fischer-Tropsch (FT) reactor by adding thermocouples, heat transfer elements, analyzers, and modifying buffer wax tanks, catalysts, regeneration units, separation units, and heating units;
modifying a hydrocracker/stripper by adding thermocouples, modifying the stripper column, adding a hydrogen makeup compressor, adding heaters, modifying specialized flow meters, and adding improved analyzers;
modifying a product fractionation system by updating internals to modify the capability of the product fractionation system to separate different grades of Gas-to-Liquid (GTL) wax along with water; and
modifying a control system and Safety Instrumented System (SIS) by modifying communication ability between analyzers and the control system, modifying the flare systems, and providing a modified effluent control system.
